(19) United States
(12) Patent Application Publication
(10) Pub. No.: US 2009/0136439 A1
(43) Pub. Date: May 28, 2009

(54) COSMETIC COMPOSITION CAPABLE OF IMPARTING EXCEPTIONAL CURL-RETENTION PROPERTIES

(75) Inventors: Sue FENG, Edison, NJ (US); Karen Yung, New York, NY (US)

Correspondence Address: L'OREAL USA/ PATENT DEPARTMENT, 30 TERMINAL AVENUE, CLARK, NJ, 07066, US

(73) Assignee: L'OREAL, Paris (FR)

(21) Appl. No.: 12/276,609

(22) Filed: Nov. 24, 2008

Related U.S. Application Data
(60) Provisional application No. 60/990,382, filed on Nov. 27, 2007.

(51) Int. Cl. A61K 8/85

(52) U.S. Cl. 424/70.7

(57) ABSTRACT

The present invention is directed to a composition and method of making-up eyelashes involving applying onto the lashes a cosmetic composition containing: (a) at least one bimodal polymer having both anionic and cationic functionalities; (b) an aqueous microdispersion of at least one wax; (c) at least one coloring agent; (d) a physiologically suitable medium, and (e) optionally, an aqueous dispersion of at least one film-forming polymer, wherein the cosmetic composition has an average particle size of less than about 10 microns.

### STATEMENT OF RELATED APPLICATIONS

This application is a non-provisional application of, and claims benefit to, U.S. Provisional Application No. 60/990,382, filed Nov. 27, 2007.

### BACKGROUND OF THE INVENTION

The present invention generally relates to a cosmetic composition used for making-up eyelashes. More particularly, by applying the cosmetic composition of the present invention onto eyelashes, long-lasting curling of the eyelashes can be achieved.

“Curling” mascara compositions having a mixture of waxes and film-forming polymers are known. Moreover, the use of organogelling agents in mascara compositions that may replace all or some of the waxes in order to achieve improved curling properties of the eyelashes are also known.

These curling mascara compositions, while they do impart a curl upon lashes treated therewith, said curl does not last for a prolonged period of time. After a relatively short period of time, the hair begins to lose its curl. Thus, the aim of the present invention is to provide a cosmetic composition capable of not only curling eyelashes, but maintaining said curl for a prolonged period of time.

### BRIEF SUMMARY OF THE INVENTION

The present invention is directed to a method of making-up eyelashes involving applying onto the lashes a cosmetic composition containing:
(a) at least one bimodal polymer having both anionic and cationic functionalities;
(b) an aqueous microdispersion of at least one wax;
(c) at least one coloring agent;
(d) a physiologically suitable medium; and
(e) optionally, an aqueous dispersion of at least one film-forming polymer,
wherein the cosmetic composition has an average particle size of less than about 10 microns.

The present invention is also directed to a composition containing:
(a) at least one bimodal polymer having both anionic and cationic functionalities;
(b) an aqueous microdispersion of at least one wax;
(c) at least one coloring agent;
(d) a physiologically suitable medium; and
(e) optionally, an aqueous dispersion of at least one film-forming polymer,
wherein the cosmetic composition has an average particle size of less than about 10 microns.

### DETAILED DESCRIPTION OF THE INVENTION

Other than in the operating examples, or where otherwise indicated, all numbers expressing quantities of ingredients and/or reaction conditions are to be understood as being modified in all instances by the term “abou”.

The present invention is directed to the surprising and unexpected discovery that by employing a bimodal interpenetrating polymer having both anionic and cationic functionalities in a cosmetic composition, wherein the composition has an average particles size of less than about 10 microns, preferably less than about 7 microns, and more preferably less than about 5 microns, the application of said composition onto lashes enables the lashes to be curled and to maintain their curl for a prolonged period of time.

The term “bimodal” describes a polymer comprised of two polymers, one having an anionic functionality and the other having a cationic functionality.

The term “physiologically suitable medium” means a non-toxic medium that can be applied to keratin fibers such as eyelashes, eyebrows and hair, and which is compatible, for instance, with a region of the eyes.

The bimodal polymer of the present invention is comprised of two distinct polymer chains, copolymerized by free-radical polymerization in a water-based system. One chain contains anionic functionality from either methacrylic acid, acrylic acid or a combination thereof. The second polymer chain contains simple amino esters of methacrylic acid or methacrylamide. The remaining monomer composition of both polymer chains is comprised of lower alkyl(C₁-C₈)esters of both methacrylic and acrylic acid.

An example of an acrylic bimodal polymer in accordance with the present invention is a styrene/acrylates/ammonium methacrylate copolymer commercially available from Interpolymer Inc. under the tradename SYNTRAN PC 5100.

The bimodal polymer will typically be present in the composition of the present invention in an amount of from about 5 to about 50% by weight, preferably from about 10 to about 35% by weight, and more preferably from about 15 to about 25% by weight, based on the total weight of the composition.

The composition of the present invention further contains an aqueous microdispersion of at least one wax. The particles of the wax microdispersion typically are of a size lower than about 1 micron, preferably lower than about 0.5 microns. These particles consist essentially of a single wax or a mixture of several waxes.

The melting point of the wax or mixture of waxes is preferably from about 50° C. to about 100° C. Moreover, the particles of the microdispersion can contain, in minor amounts, pasty or oily fatty additives, one or more surfactants and one or more conventional liposoluble active ingredients, such as those set forth below.

The composition generally contains from about 0.1 to about 40 percent by weight of the aqueous wax microdispersion, in particular from about 5 to about 30 percent by weight, more particularly from about 5 to about 15 percent by weight, and a sufficient amount of at least one emulsifying agent. The specific amount of emulsifying agent employed is that amount sufficient to obtain said wax microdispersion, such as defined above. This sufficient amount can be determined in each case by routine experimentation.

The waxes are natural substances (animal or vegetable) or synthetic materials solid at ambient temperature (20-25° C.). They are insoluble in water, soluble in oils and are capable of forming a water repellant film.

The wax or waxes may be chosen from among Carnauba wax, Candelilla wax, Alfa wax, and their mixtures.

In addition to the waxes mentioned above, the mixture of waxes can also contain one or more of the following waxes or family of waxes: paraffin wax, ozokerite, vegetable waxes such as olive tree wax, rice wax, hydrogenated jojoba wax or absolute waxes of flowers such as the essential wax of cassis flower sold by Bertin (France); animal waxes such as beeswax, or modified beeswax (cerabellina); other waxes or primary waxy materials: marine waxes such as that sold by Sophim under the name “M82”, natural or synthetic ceramides or polyethylene waxes. The vegetable waxes of Carnauba (extract of Copernica Cerifera), of Candelilla (extract of Euphobies Cerifera and Pedilantus pavonis) and of Alfa (extract of Stipa tenacissima), are commercial products.

The wax or mixture of waxes can contain, in addition to the waxes mentioned above, at least one other wax and/or at least one oil, it being understood that the mixture of waxes and optionally oil have an end melting point greater than about 50° C.

The mixture of waxes can then be combined with one or more fatty additives (oily or pasty). Mention can be made, in a non-restrictive manner, of: vegetable oils such as turnsol oil, jojoba oil, etc.; mineral oils such as paraffin oil, fluid silicone oils having a viscosity ranging principally between 0.65 and 100,000 centistokes, fluorinated oils and waxes, petrolatum and lanolin.

The mixture of oil(s) and/or pasty fatty additives can represent up to 30 percent (preferably more than 10 percent) of the weight of wax.

The use of surfactants as emulsifying agents in the preparation of wax microdispersions is known. The production of the microdispersion can be carried out using anionic, cationic and/or nonionic surfactants, in known manner.

The wax(es)/emulsifying agent(s) weight ratio can vary, for example, in the range of from about 1 to 30, and preferably from about 2 to 10.

A more detailed explanation of the types of aqueous wax microdispersions, as well as methods of making them, can be found in U.S. Pat. No. 5,849,278, the entire contents of which is hereby incorporated by reference.

The composition of the present invention may also contain an aqueous dispersion of at least one film-forming polymer, generally referred to as a latex or pseudolatex. Aqueous dispersions of film-forming polymers that may be used include the acrylic dispersions sold under the names Neocryl XK-90®, Neocryl A-1070®, Neocryl A-1090®, Neocryl BT-62®, NeocrylA-1079® and Neocryl A-523® by the company Avecia-Neoresins; Dow Latex 432® by the company Dow Chemical; Daitosol 5000 AD® by the company Daito Kasey Kogyo; Syntran 5760, Syntran 5190 and Syntran 5170 sold by the company Interpolymer; or the aqueous dispersions of polyurethane sold under the names Neorez R-981® and Neorez R-974® by the company Avecia-Neoresins; Avalure UR-405®, Avalure UR-410®, Avalure UR-425®, Avalure UR-450®, Sancure 875®, Sancure 861®, Sancure 878®, Sancure 2060® by the company Goodrich; Impranil 85® by the company Bayer and Aquamere H-1511® by the company Hydromer. Aqueous dispersions of film-forming polymers that may also be used include the polymer dispersions resulting from the free-radical polymerization of at least one free-radical monomers inside and/or partially at the surface of pre-existing particles of at least one polymer chosen from polyurethanes, polyureas, polyesters, polyesteramides and alkyd polymers. These polymers are generally referred to as hybrid polymers.

The aqueous dispersion of film forming polymer may be present in an amount of from about 0.1 to about 50% by weight, preferably from about 1 to about 25% by weight, and more preferably from about 5 to about 15% by weight, based on the total weight of the composition.

With respect to the coloring agent employed in the composition of the present invention, any pigment, dye and/or colorant capable of imparting color may be employed without departing from the spirit of the invention. The precise amount, and type, of coloring agent will depend on the desired appearance sought to be achieved and thus can be easily determined by those of ordinary skill in the cosmetic arts.

The physiologically suitable medium of the composition can essentially comprise water. It can also comprise a mixture of water and one or more water-miscible solvents, such as lower C1-5 monoalcohols, C3-C4 ketones, or C3-C4 aldehydes. The water-miscible solvent preferably used is ethanol. The content of water-miscible solvent can range from about 0.1% to about 15% by weight, preferably from about 1% to about 8% by weight, based on the total weight of the composition.

The cosmetic composition of the present disclosure may also comprise at least one additive conventionally used in cosmetics, such as antioxidants, preserving agents, fragrances, neutralizers, plasticizers, cosmetic active agents, for instance emollients, moisturizing agents, vitamins and sunscreens, and mixtures thereof. The at least one additive may be present in the composition in an amount ranging from 0.01% to 10% relative to the total weight of the composition.

Needless to say, a person skilled in the art will take care to select the optional additional additives and/or the amount thereof such that the advantageous properties of the composition according to the present disclosure are not adversely affected by the envisaged addition.

The cosmetic composition according to the present disclosure may be manufactured by the known processes generally used in cosmetics.

It has also been surprisingly and unexpectedly discovered that an even more pronounced, long-lasting curl may be achieved by an end-user when the composition is used in combination with heat.

The heating of the composition may be performed either before or after application of the product onto the eyelashes. For example, the reservoir in which the composition is contained may be equipped with heating means arranged in a manner which heats the product contained therein. Alternatively, the heating may take place via microwaves or a water bath.

On the other hand, the heating of the composition may be performed after application of the product using, for example, a conventional eyelash curling iron.

The present invention will be better understood from the examples which follow, all of which are intended for illustrative purposes only, and are not intended to unduly limit the scope of the invention in any way.

### EXAMPLES

### Example 1

### Composition having Particle Size<10 microns

| Phase | INCI NAME | % |
|---|---|---|
| A | PEG-8 Beeswax | 14 |
| | Copernicia Cerifera (Carnauba) Wax | 6 |
| | (and) PEG-14 Stearate (and) Ceteth-20 | |
| B | D.I. Water | 20 |
| | Nanosperse Ink Black PV AQII (Black | 10 |
| | 2) | |
| | Hydroxyethylcellulose | 0.35 |
| | Propylene Glycol | 5 |
| C | Copernicia Cerifera (Carnauba) Wax | 10.3 |
| | (and) PEG-30 Glyceryl Stearate | |
| D | Polyacrylate-21 (and) Acrylates/ | 21.58 |
| | Dimethylamino-ethyl Methacrylate | |
| | Copolymer (Syntran PC 5100) | |
| E | Styrene/Acrylates/Ammonium | 8.87 |
| | Methacrylate Copolymer (and) Butylene | |
| | Glycol (and) Sodium Laureth-12 | |
| | Sulfate (Syntran 5760) | |
| F | PEG/PPG-18/18 Dimethicone | 1 |
| G | Phenoxyethanol (and) Methylparaben | 0.9 |
| | (and) Isopropylparaben (and) | |
| | Isobutylparaben (and) Butylparaben | |
| H | Alcohol Denatured | 2 |

| | |
|---|---|
| TOTAL | 100 |

TABLE 1
Degree of Lash Lift Before Treatment, after Initial Application,
and 8 Hours after treatment with composition of Example 1

| | | Average Percent |
|---|---|---|
| | Angle | Change |
| Time Point | ( [symbol] °) | (%) |
| Baseline | 6.73 | — |
| Initial | 2.69 | 86.6 |
| (after application | | |
| of composition and | | |
| treatment with a | | |
| heated curler) | | |
| 8 Hours later | 4.56 | 48 |

1. In Table 1, the baseline measured between the two lines described above immediately after mascara application was 6.73°.
2. After being treated with the curler, the lashes were lifted up closer to the reference line, decreasing the angle to 2.69°.
3. After eight hours, the lashes dropped away from the fixed reference line, increasing the angle to 4.56°.

### COMPARATIVE EXAMPLE

### Composition Having Particle Size>10 microns

| Phase | INCI Name | Concentration |
|---|---|---|
| A1 | Water | 50.5 |
| A1 | Methylparaben | 0.22 |
| A1 | Hydroxyethyl | 0.22 |
| | cellulose | |
| A1 | Triethanolamine | 2.4 |
| A2 | Black Iron Oxide | 8 |
| B1 | Stearic Acid | 5.45 |
| B1 | Rice Bran Wax | 7.45 |
| B1 | Carnauba Wax | 7.3 |
| B1 | Hydrogenated | 6.3 |
| | Stearyl Olive | |
| | Esters | |
| B1 | Candelilla Cera | 2.5 |
| B1 | Propylparaben | 0.2 |
| B1 | Simethicone | 0.12 |
| B1 | PPG-17/IPDI/PDMPA | 0.1 |
| | Copolymer | |
| B1 | BHT | 0.1 |
| B2 | Water | 1.52 |
| B2 | Acacia Senegal | 1.52 |
| C | Water | 3.7 |
| C | Polyvinyl Alcohol | 0.2 |
| D | PEG/PPG-18/18 | 0.2 |
| | Dimethicone | |
| E | Water | 1 |
| E | Panthenol | 1 |

| | |
|---|---|
| Total | 100 |

TABLE 2
Degree of Lash Lift Before Treatment, after Initial Application, and
8 Hours after treatment with Comparative example composition]

| | | Average Percent |
|---|---|---|
| | Angle | Change |
| Time Point | ( [symbol] °) | (%) |
| Baseline | 7.22 | — |
| Initial | 2.99 | 57.5 |
| (after application | | |
| of comparative | | |
| composition and | | |
| treatment with a | | |
| heated curler) | | |
| 8 Hours later | 4.62 | 32.2 |

1. In Table 2, the baseline measured between the two lines described above immediately after mascara application was 7.22°.
2. After being treated with the curler, the lashes were lifted up closer to the reference line, decreasing the angle to 2.99°.
3. After eight hours, the lashes dropped away from the fixed reference line, increasing the angle to 4.62°.
   As can be clearly seen from the above-referenced data, a composition in accordance with the present invention both surprisingly, and unexpectedly, provided greater initial and retained curl to eyelashes treated therewith, as compared to a conventional mascara composition.

What is claimed is:

1. A method of making-up eyelashes comprising applying onto the lashes a cosmetic composition containing:
(a) at least one bimodal polymer having both anionic and cationic functionalities;
(b) an aqueous microdispersion of at least one wax;
(c) at least one coloring agent;
(d) a physiologically suitable medium; and
(e) optionally, an aqueous dispersion of at least one film-forming polymer,
wherein the cosmetic composition has an average particle size of less than about 10 microns.

2. The method of claim 1 wherein (a) is a styrene/acrylates/ammonium methacrylate copolymer.

3. The method of claim 1 wherein (a) is present in the composition in an amount of from about 5 to about 50% by weight, based on the weight of the composition.

4. The method of claim 1 wherein (a) is present in the composition in an amount of from about 15 to about 25% by weight, based on the weight of the composition.

5. The method of claim 1 wherein (b) is at least one wax chosen from Carnauba, Candellila and Alfa.

6. The method of claim 1 wherein (b) is present in the composition in an amount of from about 0.1 to about 40% by weight, based on the weight of the composition.

7. The method of claim 1 wherein (b) is present in the composition in an amount of from about 5 to about 15% by weight, based on the weight of the composition.

8. The method of claim 1 wherein the composition has an average particle size of less than about 5 microns.

9. The method of claim 1 wherein (e) is present in the composition in an amount of from about 0.1 to about 50% by weight, based on the weight of the composition.

10. The method of claim 1 wherein (e) is present in the composition in an amount of from about 5 to about 15% by weight, based on the weight of the composition.

11. The method of claim 1 further comprising heating the cosmetic composition prior to applying it onto the eyelashes.

12. The method of claim 1 further comprising heating the cosmetic composition after applying it onto the eyelashes.

13. A cosmetic composition comprising:
(a) at least one bimodal polymer having both anionic and cationic functionalities;
(b) an aqueous microdispersion of at least one wax;
(c) at least one coloring agent;
(d) a physiologically suitable medium; and
(e) optionally, an aqueous dispersion of at least one film-forming polymer,
wherein the cosmetic composition has an average particle size of less than about 10 microns.

14. The composition of claim 13 wherein (a) is a styrene/acrylates/ammonium methacrylate copolymer.

15. The composition of claim 13 wherein (a) is present in the composition in an amount of from about 5 to about 50% by weight, based on the weight of the composition.

16. The composition of claim 13 wherein (a) is present in the composition in an amount of from about 15 to about 25% by weight, based on the weight of the composition.

17. The composition of claim 13 wherein (b) is at least one wax chosen from Carnauba, Candellila and Alfa.

18. The composition of claim 13 wherein (b) is present in the composition in an amount of from about 0.1 to about 40% by weight, based on the weight of the composition.

19. The composition of claim 13 wherein (b) is present in the composition in an amount of from about 5 to about 15% by weight, based on the weight of the composition.

20. The composition of claim 13 wherein the composition has an average particle size of less than about 5 microns.

21. The composition of claim 13 wherein (e) is present in the composition in an amount of from about 0.1 to about 50% by weight, based on the weight of the composition.

22. The composition of claim 13 wherein (e) is present in the composition in an amount of from about 5 to about 15% by weight, based on the weight of the composition.
